(19) United States
(12) Patent Application Publication
(10) Pub. No.: US 2024/0189285 A1
(43) Pub. Date: Jun. 13, 2024

(54) COMPOSITION AND METHOD FOR ENHANCING MITOCHONDRIAL BIOGENESIS AND ACTIVATING PGC-1A

(71) Applicant: NANJING NUTRABUILDING BIO-TECH CO., LTD., Nanjing (CN)

(72) Inventors: Mingru WANG, Nanjing (CN); Ronghua YI, Nanjing (CN); Shawn WELLS, Nanjing (CN); Kylin LIAO, Nanjing (CN)

(21) Appl. No.: 18/586,160

(22) Filed: Feb. 23, 2024

Related U.S. Application Data
(63) Continuation of application No. PCT/CN2022/114007, filed on Aug. 22, 2022.

(30) Foreign Application Priority Data
Aug. 26, 2021 (WO) PCT/CN2021/114807
Aug. 26, 2021 (WO) PCT/CN2021/114826

(51) Int. Cl. A61K 31/4172; A61P 3/00

(52) U.S. Cl. CPC A61K 31/4172; A61P 3/00

(57) ABSTRACT

The present invention provides methods and compounds for enhancing mitochondrial biogenesis in a mammal or for activating PGC-1α in a mammal, comprising administrating to the mammal in need thereof a therapeutically effective amount of L-Ergothioneine, an analog or derivative thereof, or a pharmaceutically acceptable salt, acid, ester, analog or derivative thereof (e.g., as an active ingredient). Such administration of L-Ergothioneine is also capable of alleviating mitochondrial dysfunction and boosting cellular function by enhancing mitochondrial biogenesis, and/or promoting health and longevity by improving mitochondrial biogenesis.

### CROSS REFERENCE TO RELATED APPLICATION

This application is a continuation application of International Patent Application No. PCT/CN2022/114007, filed on Aug. 22, 2022, which claims the priority of the international application Nos. PCT/CN2021/114826 and PCT/CN2021/114807, both filed on Aug. 26, 2021, the contents of all of which are incorporated herein by reference in their entirety.

### FIELD OF THE INVENTION

This invention generally relates to the field of methods for enhancing mitochondrial biogenesis in a mammal and the field of PGC-1α activation in a mammal, and more specifically relates to compositions and methods for enhancing mitochondrial biogenesis by activating PGC-1α or relates to compositions and methods for PGC-1α activation, involving administrating to a mammal in need thereof a therapeutically effective amount of: L-Ergothioneine, an analog or derivative thereof, or a pharmaceutically acceptable salt, acid, ester, analog or derivative thereof.

### BACKGROUND OF THE INVENTION

Mitochondrial biogenesis is the cellular process that produces new mitochondria—which is one of the ways that cells adapt to the ever-changing energy requirements dictated by environmental and physiological conditions. Under circumstances of decreased fuel supply (e.g., caloric restriction) or increased energy demand (e.g., exercise), cells need to adjust their metabolic processes in order to maintain the availability of cellular energy (as ATP) to meet the energetic needs of tissues. An age-related decline in mitochondrial biogenesis has been shown to be associated with a loss of mitochondrial mass and a decline in mitochondrial function, particularly in high energy-demanding tissues such as skeletal muscle, heart, and brain. On the other hand, increased mitochondrial biogenesis can alleviate mitochondrial dysfunction and boost cellular function. The pathways that regulate mitochondrial biogenesis are known to affect longevity, and there are a number of interventions that have been shown to promote health and longevity by (among other effects) improving mitochondrial biogenesis. An age-associated decline of PGC-1α levels may be among the main causes of loss of mitochondrial biogenesis capacity. Therefore, supporting the expression, activation and activity of PGC-1α is a key target in the support of mitochondrial biogenesis.

Peroxisome proliferator-activated receptor γ co-activator 1α (PGC-1α) is a well-known controlling factor of energy metabolism in the body. PGC-1α was originally identified as a transcription coupling factor in brown adipose tissue that activates transcription dependent on the nuclear receptor PPARγ. Then it has been found that that not only PPARγ, but also PGC-1α interacts with many nuclear receptors or various transcription factors, which are involved in a wide variety of biological responses, such as adaptive thermogenesis, mitochondrial biogenesis, glucose/fatty acid metabolism, fiber type switching in skeletal muscle, and heart development.

The expression and activity of PGC-1α is regulated by both cell-autonomous factors (e.g., AMP-activated kinase (AMPK) and mTOR) and systemic factors of hormonal origin, such as insulin, glucagon, glucocorticoids and centrally regulated neural outputs. In addition to the regulation of its expression level, PGC-1α activity is also controlled by a variety of posttranslational modifications, including phosphorylation, ubiquitination and acetylation.

By far, sirtuin (Sirt1) is the only protein identified to be able to deacetylate PGC-1α. Sirt1 belongs to a family of class III histone/protein deacetylase proteins, including seven members in mammals (i.e., Sirt1-Sirt7), with different cellular localizations. Sirt1 is the mammalian homolog of the Silent information regulator 2 (Sir2), which was initially identified as a trans-acting factor involved in repression of the silent mating-type loci in yeast. Sirt1 is the most studied mammalian orthologue and, as described above, is responsible for deacetylation of PGC-1α. Unlike histone deacetylation, which condenses chromatin structure and attenuates general transcriptional activity, PGC-1α deacetylation enhances its activity to co-activate transcription factors that in turn will induce transcription of its target genes.

L-Ergothioneine is a water-soluble amino acid found mainly in mushrooms, but also in king crab, meat from animals that have grazed on grasses containing L-Ergothioneine, and other foods. L-Ergothioneine cannot be synthesized by the human body (or other vertebrates), as it can only be supplemented by diet. This compound has a wide tissue distribution in human, with a more abundant accumulation in erythrocytes, liver, seminal fluid, bone marrow, eye lens, cornea, and kidney. L-Ergothioneine is a powerful energy-boosting antioxidant. It is a naturally occurring thiol/thione derivative of the essential amino acid histidine, its molecular formula is C₉H₁₅N₃O₂S—combined with hydrogen, that sulfur at the end is what actually classifies it as a “thiol”, the “—SH group”, is either referred to as a thiol group or a sulfanyl group. Thiols have a strong affinity towards soft metals, which contributes to L-Ergothioneine's scavenging effects.

It is therefore desired to have novel and effective compounds and methods for enhancing mitochondrial biogenesis or PGC-1α activation.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

The present invention generally relates to compounds and methods for enhancing mitochondrial biogenesis in a mammal (e.g., animals or human), comprising administrating to the mammal in need thereof a therapeutically effective amount of L-Ergothioneine, an analog or derivative thereof, or a pharmaceutically acceptable salt, acid, ester, analog or derivative thereof (e.g., as an active ingredient). In particular, it was surprisingly found that L-Ergothioneine can enhance mitochondrial biogenesis by activating PGC-1α. Moreover, the administration of L-Ergothioneine is further capable of alleviating mitochondrial dysfunction and boosting cellular function by enhancing mitochondrial biogenesis, and capable of promoting health and longevity by improving mitochondrial biogenesis.

One aspect of the present invention provides a method for enhancing mitochondrial biogenesis in a mammal, comprising administrating to the mammal in need thereof a therapeutically effective amount of L-Ergothioneine, an analog or derivative thereof, or a pharmaceutically acceptable salt, acid, ester, analog or derivative thereof, as an active ingredient.

In some embodiments, L-Ergothioneine is administrated to enhance mitochondrial biogenesis by activating PGC-1α.

In some embodiments, the administration of L-Ergothioneine is configured for alleviating mitochondrial dysfunction and boosting cellular function by enhancing mitochondrial biogenesis.

In some embodiments, the administration of L-Ergothioneine is configured for promoting health and longevity by improving mitochondrial biogenesis.

In some embodiments, L-Ergothioneine is administrated in an amount ranging from 0.1 μM to 1 M (e.g., 0.1 μM to 500 μM, 1 μM to 500 μM, 1 μM to 5 mM, 1 μM to 100 mM, 1 μM to 500 mM, 5 μM to 500 μM, 5 μM to 5 mM, 5 μM to 100 mM, 5 μM to 500 mM, 50 μM to 500 μM, 50 μM to 5 mM).

In some embodiments, L-Ergothioneine is administrated in an amount ranging from 2-2000 mg, 2-500 mg, 2-200 mg, 2-150 mg, 5-100 mg, or 5-50 mg.

Still in some embodiments, L-Ergothioneine is prepared in a form of nutritional, drinking, or pharmaceutical composition, for use in a food, drink, nutritional or pharmaceutical products.

In some embodiments, L-Ergothioneine is administrated as a dietary supplement or an ingredient in a food.

In some embodiments, L-Ergothioneine is administrated in a form of solutions, liquid suspensions, parenteral solutions, injections, tablets, pills, granules, powder, film, (micro)capsules, aerosols, tonics, syrups, beverages, or nourishments.

In some embodiments, L-Ergothioneine may be administrated orally, by intravenous injection, by intramuscular injection, intraperitoneally or sublingually. For instance, L-Ergothioneine may be administrated in a daily dose ranging from 0.1 μM to 1 M (e.g., 0.1 μM to 500 μM, 1 μM to 500 μM, 1 μM to 5 mM, 1 μM to 100 mM, 1 μM to 500 mM, 5 μM to 500 μM, 5 μM to 5 mM, 5 μM to 100 mM, 5 μM to 500 mM, 50 μM to 500 μM, 50 μM to 5 mM), by either a single dose or multiple divided doses. In some embodiments, the administration of the composition is by oral with a daily dose of ergothioneine in the range of 2-2000 mg, 2-500 mg, 2-200 mg, 2-150 mg, 5-100 mg, or 5-50 mg.

Another aspect of the present invention relates to a composition for enhancing mitochondrial biogenesis in a mammal, comprising a therapeutically effective amount of L-Ergothioneine, an analog or derivative thereof, or a pharmaceutically acceptable salt, acid, ester, analog or derivative thereof, wherein L-Ergothioneine effectively enhances mitochondrial biogenesis.

In some embodiments, the composition enhances mitochondrial biogenesis by activating PGC-1α.

In some embodiments, the composition alleviates mitochondrial dysfunction and boosts cellular function by enhancing mitochondrial biogenesis.

In some embodiments, the composition promotes health and longevity by improving mitochondrial biogenesis.

In some embodiments, L-Ergothioneine is in an amount ranging from 0.1 μM to 1 M (e.g., 0.1 μM to 500 μM, 1 μM to 500 μM, 1 μM to 5 mM, 1 μM to 100 mM, 1 μM to 500 mM, 5 μM to 500 μM, 5 μM to 5 mM, 5 μM to 100 mM, 5 μM to 500 mM, 50 μM to 500 μM, 50 μM to 5 mM).

In some embodiments, L-Ergothioneine is administrated in an amount ranging from 2-2000 mg, 2-500 mg, 2-200 mg, 2-150 mg, 5-100 mg, or 5-50 mg.

A further aspect of the present invention provides use of L-Ergothioneine in manufacturing a composition for enhancing mitochondrial biogenesis in a mammal.

In some embodiments, L-Ergothioneine enhances mitochondrial biogenesis by activating PGC-1α.

In some embodiments, L-Ergothioneine alleviates mitochondrial dysfunction and boosts cellular function by enhancing mitochondrial biogenesis.

In some embodiments, L-Ergothioneine promotes health and longevity by improving mitochondrial biogenesis.

In some embodiments, L-Ergothioneine is in an amount ranging from 0.1 μM to 1 M (e.g., 0.1 μM to 500 μM, 1 μM to 500 μM, 1 μM to 5 mM, 1 μM to 100 mM, 1 μM to 500 mM, 5 μM to 500 μM, 5 μM to 5 mM, 5 μM to 100 mM, 5 μM to 500 mM, 50 μM to 500M, 50 μM to 5 mM).

In some embodiments, L-Ergothioneine is administrated in an amount ranging from 2-2000 mg, 2-500 mg, 2-200 mg, 2-150 mg, 5-100 mg, or 5-50 mg.

In some embodiments, L-Ergothioneine is in a form of solutions, liquid suspensions, parenteral solutions, injections, tablets, pills, granules, powder, film, (micro)capsules, aerosols, tonics, syrups, beverages, or nourishments.

In still a further aspect, the present invention provides a method of preparing a composition for enhancing mitochondrial biogenesis in a mammal, comprising a step of preparing an effective amount of L-Ergothioneine, an analog or derivative thereof, or a pharmaceutically acceptable salt, acid, ester, analog or derivative thereof.

In some embodiments, L-Ergothioneine enhances mitochondrial biogenesis by activating PGC-1α.

In some embodiments, the composition alleviates mitochondrial dysfunction and boosts cellular function by enhancing mitochondrial biogenesis, and/or promotes health and longevity by improving mitochondrial biogenesis.

In some embodiments, L-Ergothioneine is prepared in an amount ranging from 0.1 μM to 1 M (e.g., 0.1 μM to 500 μM, 1 μM to 500 μM, 1 μM to 5 mM, 1 μM to 100 mM, 1 μM to 500 mM, 5 μM to 500 μM, 5 μM to 5 mM, 5 μM to 100 mM, 5 μM to 500 mM, 50 μM to 500 μM, 50 μM to 5 mM).

In some embodiments, L-Ergothioneine is administrated in an amount ranging from 2-2000 mg, 2-500 mg, 2-200 mg, 2-150 mg, 5-100 mg, or 5-50 mg.

Still in some embodiments, L-Ergothioneine is prepared in a form of solutions, liquid suspensions, parenteral solutions, injections, tablets, pills, granules, powder, film, (micro)capsules, aerosols, tonics, syrups, beverages, or nourishments.

The present invention generally relates to compounds and methods for activating PGC-1α in a mammal (e.g., animals or human), comprising administrating to the mammal in need thereof a therapeutically effective amount of L-Ergothioneine, an analog or derivative thereof, or a pharmaceutically acceptable salt, acid, ester, analog or derivative thereof (e.g., as an active ingredient). Due to the outstanding antioxidant capacity of L-Ergothioneine, it can protect mitochondrial function and protect gene stability, etc. According to the present invention, it was surprisingly found that L-Ergothioneine can activate PGC-1α by upregulating the Sirt1 expression. It is believed that this invention is the first time to propose and conduct L-Ergothioneine as an active ingredient to activate PGC-1α.

One aspect of the present invention provides a method for activating PGC-1α in a mammal, comprising administrating to the mammal in need thereof a therapeutically effective amount of L-Ergothioneine, or a pharmaceutically acceptable salt, acid, ester, analog or derivative thereof, as an active ingredient.

In some embodiments, L-Ergothioneine is administrated to upregulate Sirt1 expression in the mammal.

In some embodiments, L-Ergothioneine is administrated in an amount ranging from 0.1 μM to 1 M (e.g., 0.1 μM to 500 μM, 1 μM to 500 μM, 1 μM to 5 mM, 1 μM to 100 mM, 1 μM to 500 mM, 5 μM to 500 μM, 5 μM to 5 mM, 5 μM to 100 mM, 5 μM to 500 mM, 50 μM to 500 μM, 50 μM to 5 mM).

In some embodiments, L-Ergothioneine is administrated in an amount ranging from 2-2000 mg, 2-500 mg, 2-200 mg, 2-150 mg, 5-100 mg, or 5-50 mg.

In some embodiments, L-Ergothioneine is prepared in a form of nutritional, drinking, or pharmaceutical composition, for use in a food, drink, nutritional or pharmaceutical products.

In some embodiments, L-Ergothioneine is administrated as a dietary supplement or an ingredient in a food.

In some embodiments, L-Ergothioneine is administrated in a form of solutions, liquid suspensions, parenteral solutions, injections, tablets, pills, granules, powder, film, (micro)capsules, aerosols, tonics, syrups, beverages, or nourishments.

In some embodiments, L-Ergothioneine is administrated orally, by intravenous injection, by intramuscular injection, intraperitoneally or sublingually.

In some embodiments, L-Ergothioneine is administrated is administrated in a daily dose ranging from 0.1 μM to 1 M (e.g.,0.1 to 500 μM), by a single dose or multiple divided doses. In some embodiments, the administration of the composition is by oral with a daily dose of ergothioneine in the range of 2-2000 mg, 2-500 mg, 2-200 mg, 2-150 mg, 5-100 mg, or 5-50 mg.

Another aspect of this invention relates to a composition for activating PGC-1α in a mammal, comprising a therapeutically effective amount of L-Ergothioneine, an analog or derivative thereof, or a pharmaceutically acceptable salt, acid, ester, analog or derivative thereof, wherein L-Ergothioneine effectively activates PGC-1α.

In some embodiments, L-Ergothioneine effectively upregulates Sirt1 expression in the mammal.

In some embodiments, L-Ergothioneine is in an amount ranging from 0.1 μM to 1 M (e.g., 0.1 μM to 500 μM, 1 μM to 500 μM, 1 μM to 5 mM, 1 μM to 100 mM, 1 μM to 500 mM, 5 μM to 500 μM, 5 μM to 5 mM, 5 μM to 100 mM, 5 μM to 500 mM, 50 μM to 500 μM, 50 μM to 5 mM).

In some embodiments, L-Ergothioneine is administrated in an amount ranging from 2-2000 mg, 2-500 mg, 2-200 mg, 2-150 mg, 5-100 mg, or 5-50 mg.

In some embodiments, L-Ergothioneine is in a form of solutions, liquid suspensions, parenteral solutions, injections, tablets, pills, granules, powder, film, (micro)capsules, aerosols, tonics, syrups, beverages, or nourishments.

In some embodiments, the composition is to be administrated into a mammal. In some further embodiments, the composition may be administrated orally, by intravenous injection, by intramuscular injection, intraperitoneally or sublingually. For instance, the composition may be administrated as a dietary supplement or an ingredient in a food.

A further aspect of the present invention relates to use of L-Ergothioneine in manufacturing a composition for activating PGC-1α in a mammal.

In some embodiments, L-Ergothioneine upregulates the Sirt1 expression in the mammal.

In some embodiments, L-Ergothioneine is in an amount ranging from 0.1 μM to 1 M (e.g., 0.1 μM to 500 μM, 1 μM to 500 μM, 1 μM to 5 mM, 1 μM to 100 mM, 1 μM to 500 mM, 5 μM to 500 μM, 5 μM to 5 mM, 5 μM to 100 mM, 5 μM to 500 mM, 50 μM to 500 μM, 50 μM to 5 mM).

In some embodiments, L-Ergothioneine is administrated in an amount ranging from 2-2000 mg, 2-500 mg, 2-200 mg, 2-150 mg, 5-100 mg, or 5-50 mg.

In some embodiments, L-Ergothioneine is in a form of solutions, liquid suspensions, parenteral solutions, injections, tablets, pills, granules, powder, film, (micro)capsules, aerosols, tonics, syrups, beverages, or nourishments.

Still in a further aspect, the present invention provides a method of preparing a composition for activating PGC-1α in a mammal, comprising a step of preparing an effective amount of L-Ergothioneine, an analog or derivative thereof, or a pharmaceutically acceptable salt, acid, ester, analog or derivative thereof.

In some embodiments, L-Ergothioneine upregulates the Sirt1 expression in the mammal.

In some embodiments, L-Ergothioneine is prepared in an amount ranging from 0.1 μM to 1 M (e.g., 0.1 μM to 500 μM, 1 μM to 500 μM, 1 μM to 5 mM, 1 μM to 100 mM, 1 μM to 500 mM, 5 μM to 500 μM, 5 μM to 5 mM, 5 μM to 100 mM, 5 μM to 500 mM, 50 μM to 500 μM, 50 μM to 5 mM). In some embodiments, L-Ergothioneine is prepared in a form of solutions, liquid suspensions, parenteral solutions, injections, tablets, pills, granules, powder, film, (micro)capsules, aerosols, tonics, syrups, beverages, or nourishments.

### BRIEF DESCRIPTION OF THE DRAWINGS

FIG. 1 is a Western blot of PGC-1α protein expression of A, B, C and D group cells.

FIG. 2 is a Western blot of Sirt1 protein expression of A, B, C and D group cells.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

The terms ‘comprising’ or ‘to comprise’ and their conjugations, as used herein, refer to a situation wherein said terms are used in their non-limiting sense to mean that items following the word are included, but items not specifically mentioned are not excluded. It also encompasses the more limiting verb ‘to consist essentially of’ and ‘to consist of’.

As used herein “animal,” “mammal,” may be used interchangeably to refer to any mammal to which the presently disclosed methods and compositions may be applied or administered. The mammal may have an illness or other disease, but the mammal does not need to be sick to benefit from the presently disclosed methods and compositions. As such any mammal may consume the disclosed compositions or be a recipient of the disclosed methods. More typically, a mammal as referred to herein includes a human or a domesticated animal. For example, domesticated animals include a dog, cat, or any farm animal including horses, cows, sheep, goats, pigs, or chickens.

The term “administer”, “administering”, or “administration”, as used in this invention, refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

“Pharmaceutically acceptable salts” refers to those salts which retain the biological effectiveness and properties of the free bases and which are obtained by reaction with inorganic acids, for example, acid, hydrobromic acid, sulfuric acid, nitric acid, and phosphoric acid or organic acids such as sulfonic acid, carboxylic acid, organic phosphoric acid, methanesulfonic acid, ethanesulfonic acid, toluenesulfonic acid, citric acid, fumaric acid, maleic acid, succinic acid, benzoic acid, salicylic acid, lactic acid, tartaric acid (e, (+) or (−)-tartaric acid or mixtures thereof), amino acids (e.g., (+) or (−)-amino acids or mixtures thereof), and the like. These salts can be prepared by methods known to those skilled in the art. “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art.

The term “effective amount” means an amount of a compound according to the invention which, in the context of which it is administered or used, is sufficient to achieve the desired effect or result. Depending on the context, the term effective amount may include or be synonymous with a pharmaceutically effective amount or a therapeutically effective amount. An effective amount can be determined by methods known to those of skill in the art.

Depending on the particular L-Ergothioneine formulation and form, contemplated methods include an administration of an amount ranging from 0.1 μM to 1 M (e.g., 0.1 μM to 500 μM, 1 μM to 500 μM, 1 μM to 5 mM, 1 μM to 100 mM, 1 μM to 500 mM, 5UM to 500μM, 5 μM to 5 mM, 5 μM to 100 mM, 5 μM to 500 mM, 50 μM to 500 μM, 50 μM to 5 mM) of L-Ergothioneine. This amount per day may be administered at once or in multiple doses. Typically, an amount of L-Ergothioneine to be administered at once or in multiple doses per day is of 0.1 μM to 1 M (e.g., 0.1 μM to 500 μM, 1 μM to 500M, 1 μM to 5 mM, 1 μM to 100 mM, 1 μM to 500 mM, 5 μM to 500 μM, 5pM to 5 mM, 5 μM to 100 mM, 5 μM to 500 mM, 50 μM to 500 μM, 50 μM to 5 mM). The dose or doses may be administered once a day for any period of time. For example, the effective dose may be administered each day for one day, a few days, multiple days, or on a daily basis indefinitely. Typically, an amount of L-Ergothioneine to be administered at once or in multiple doses per day is of or between 2-2000 mg, 2-500 mg, 2-200 mg, 2-150 mg, 5-100 mg, or 5-50 mg.

Generally speaking, various embodiments of the present invention provide for compounds and methods for enhancing mitochondrial biogenesis by activating PGC-1α, alleviating mitochondrial dysfunction and boosting cellular function by enhancing mitochondrial biogenesis, and/or promoting health and longevity by improving mitochondrial biogenesis—comprising administration of a therapeutically effective amount of L-Ergothioneine, an analog or derivative thereof, or a pharmaceutically acceptable salt, acid, ester, analog or derivative thereof (e.g., as an active ingredient) in a mammal (e.g., animals or human) in need thereof. Particularly, L-Ergothioneine can activate PGC-1α by upregulating the Sirt1 expression. Moreover, L-Ergothioneine may be prepared or administrated in a variety of forms, such as solutions, liquid suspensions, parenteral solutions, injections, tablets, pills, granules, powder, film, (micro)capsules, aerosols, tonics, syrups, beverages, and nourishments. For instance, L-Ergothioneine may be prepared or administrated (e.g., by oral) with an amount ranging from 0.1 μM to 1 M (e.g., 0.1 μM to 500 μM, 1 μM to 500 μM, 1 μM to 5 mM, 1 μM to 100 mM, 1 μM to 500 mM, 5 μM to 500 μM, 5UM to 5 mM, 5 μM to 100 mM, 5 μM to 500 mM, 50 μM to 500 μM, 50 μM to 5 mM). Further, the present invention also provides use of L-Ergothioneine in manufacturing a composition for activating PGC-1α in a mammal, as well a method of preparing a composition for activating PGC-1α in a mammal, comprising a step of preparing an effective amount of L-Ergothioneine, an analog or derivative thereof, or a pharmaceutically acceptable salt, acid, ester, analog or derivative thereof.

Various embodiments of the present invention provide for compounds and methods for activating PGC-1α in a mammal (e.g., animal or human), involving inclusion/administration of a therapeutically effective amount of L-Ergothioneine, an analog or derivative thereof, or a pharmaceutically acceptable salt, acid, ester, analog or derivative thereof, e.g., as an active ingredient. Particularly, L-Ergothioneine can activate PGC-1α by upregulating the Sirt1 expression. Moreover, L-Ergothioneine may be prepared or administrated in a variety of forms, such as solutions, liquid suspensions, parenteral solutions, injections, tablets, pills, granules, powder, film, (micro)capsules, aerosols, tonics, syrups, beverages, and nourishments. For instance, L-Ergothioneine may be prepared or administrated (e.g., by oral) with an amount ranging from 0.1 μM to 1 M (e.g., 0.1 μM to 500 μM, 1 μM to 500 μM, 1 μM to 5 mM, 1 μM to 100 mM, 1 μM to 500 mM, 5 μM to 500 μM, 5 μM to 5 mM, 5 μM to 100 mM, 5 μM to 500 mM, 50 μM to 500 μM, 50 μM to 5 mM). Further, the present invention also provides use of L-Ergothioneine in manufacturing a composition for activating PGC-1α in a mammal, as well a method of preparing a composition for activating PGC-1α in a mammal, comprising a step of preparing an effective amount of L-Ergothioneine, an analog or derivative thereof, or a pharmaceutically acceptable salt, acid, ester, analog or derivative thereof.

The following examples are illustrative of select embodiments of the present invention and are not meant to limit the scope of the invention.

### EXAMPLE

Target cells (endothelial cells, C2C12 skeletal muscle cells, etc.) are obtained according to conventional methods in the art.

In vitro tests were conducted to demonstrate that L-Ergothioneine can enhance mitochondrial biogenesis. The target cells (endothelial cells, C2C12 skeletal muscle cells, etc.) were divided into groups A, B, C and D. B and D group cells were cultured with 50 μM L-Ergothioneine for 12 hours. A and C groups were not cultured with Ergothioneine but only with the usual nutrient solution for 12 hours. Then a certain concentration of H₂O₂ was used to cause C and D group cells damage, no H₂O₂ was used on A and B groups. Mitochondrial biogenesis was measured by quantifying both mitochondrial mass and mitochondrial DNA (mtDNA) contents. Mitochondrial mass was measured by flow cytometry and fluorescent microscopy, mtDNA copy number was analyzed by quantitative PCR.

TABLE 1

| | Relative mitochondrial mass | Relative mtDNA content |
|---|---|---|
| | percent, % | percent, % |
| A | 100 | 100 |
| B | 112 | 118 |
| C | 78 | 89 |
| D | 90 | 93 |

Table 1 shows the results of relative mitochondrial mass and relative mtDNA contents of the four groups. Relative mitochondrial mass and relative mtDNA contents were increased significantly in B group (cultured with L-Ergothioneine) compared to the A group, both of them were not treated with H₂O₂. Relative mitochondrial mass and relative mtDNA contents were higher in D group (cultured with L-Ergothioneine) than the C group, cells damage was caused in both C and D groups by H₂O₂ treatment. The results show that L-Ergothioneine can enhance mitochondrial biogenesis.

In vitro tests were conducted to demonstrate that L-Ergothioneine can activate PGC-1α. The target cells (endothelial cells, C₂C₁₂ skeletal muscle cells, etc.) are divided into groups A, B, C and D. B and D group cells were cultured with 50 μM L-Ergothioneine for 12 hours, and A and C groups were not cultured with Ergothioneine but only with the usual nutrient solution for 12 hours. Then a certain concentration of H₂O₂ was used to cause C and D group cells damage, no H₂O₂ was used on A and B groups. Then PGC-1α protein expression and Sirt1 protein level were analyzed by Western blot.

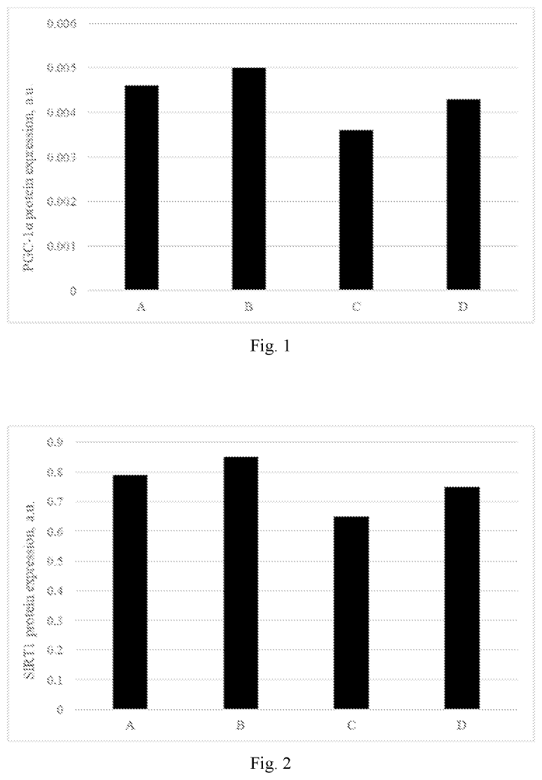

FIG. 1 is a Western blot of PGC-1α protein expression of A, B, C and D group cells. The PGC-1α levels in A, B, C and D groups were about 0.0046, 0.005, 0.0036, 0.0043 respectively. PGC-1α level was higher in B group (cultured with L-Ergothioneine) than the A group, both of them were not treated with H₂O₂. PGC-1α level was higher in D group (cultured with L-Ergothioneine) than the C group, cells damage was caused in both C and D groups by H₂O₂ treatment. FIG. 2 is a Western blot of Sirt1 protein expression of A, B, C and D group cells. The Sirt1 levels in A, B, C and D groups were about 0.79, 0.85, 0.65, 0.75 respectively. Sirt1 level was higher in B group (cultured with L-Ergothioneine) than the A group, both of them were not treated with H₂O₂. Sirt1 level was higher in D group (cultured with L-Ergothioneine) than the C group, b cells damage was caused in both C and D groups by H₂O₂ treatment. The results show that L-Ergothioneine can activate PGC-1α and upregulate Sirt1 expression in target cells.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

### Other Embodiments

It is to be understood that while the invention has been described in conjunction with the detailed description thereof and accompanying figures, the foregoing description and accompanying figures are only intended to illustrate, and not limit the scope of the invention, which is defined by the scope of the appended claims. Other aspects, advantages, and modifications are within the scope of the following claims. All publications referenced herein are incorporated by reference in their entireties.

What is claimed is:

1. A method for enhancing mitochondrial biogenesis in a mammal, comprising administrating to the mammal in need thereof a therapeutically effective amount of L-Ergothioneine, an analog or derivative thereof, or a pharmaceutically acceptable salt, acid, ester, analog or derivative thereof, as an active ingredient.

2. The method of claim 1, wherein the L-Ergothioneine is administrated to enhance mitochondrial biogenesis by activating PGC-1α.

3. The method of claim 1, wherein the administration of the L-Ergothioneine is configured for alleviating mitochondrial dysfunction, boosting cellular function or promoting health and longevity by enhancing mitochondrial biogenesis.

4. The method of claim 1, wherein the L-Ergothioneine is administrated in an amount ranging from 0.1 μM to 1 M.

5. The method of claim 4, wherein the L-Ergothioneine is administrated in an amount ranging from 0.1 to 500 μM.

6. The method of claim 1, wherein the L-Ergothioneine is prepared in a form of nutritional, drinking, or pharmaceutical composition, for use in a food, drink, nutritional or pharmaceutical products.

7. The method of claim 1, wherein the L-Ergothioneine is administrated as a dietary supplement or an ingredient in a food.

8. The method of claim 1, wherein the L-Ergothioneine is administrated in a form of solutions, liquid suspensions, parenteral solutions, injections, tablets, pills, granules, powder, film, (micro)capsules, aerosols, tonics, syrups, beverages, or nourishments.

9. The method of claim 1, wherein the L-Ergothioneine is administrated orally, by intravenous injection, by intramuscular injection, intraperitoneally or sublingually.

10. The method of claim 1, wherein the L-Ergothioneine is administrated in a daily dose ranging from 0.1 μM to 1 M, or from 0.1 to 500 μM, by a single dose or multiple divided doses.

11. A composition for enhancing mitochondrial biogenesis in a mammal, comprising a therapeutically effective amount of L-Ergothioneine, an analog or derivative thereof, or a pharmaceutically acceptable salt, acid, ester, analog or derivative thereof, wherein the L-Ergothioneine effectively enhances mitochondrial biogenesis.

12. The composition of claim 11, wherein the composition enhances mitochondrial biogenesis by activating PGC-1α.

13. The composition of claim 11, wherein the composition alleviates mitochondrial dysfunction, boosts cellular function or promotes health and longevity by enhancing mitochondrial biogenesis.

14. The method of claim 10, wherein the L-Ergothioneine is in an amount ranging from 0.1 μM to 1 M.

15. Use of L-Ergothioneine in manufacturing a composition for enhancing mitochondrial biogenesis in a mammal.

16. The use of claim 15, wherein the L-Ergothioneine enhances mitochondrial biogenesis by activating PGC-1α.

17. The use of claim 15, wherein the L-Ergothioneine alleviates mitochondrial dysfunction, boosts cellular function or promotes health and longevity by enhancing mitochondrial biogenesis.

18. The use of claim 15, wherein the L-Ergothioneine promotes health and longevity by improving mitochondrial biogenesis.

19. The use of claim 15, wherein the L-Ergothioneine is in an amount ranging from 0.1 μM to 1 M.

20. The use of claim 15, wherein the L-Ergothioneine is in a form of solutions, liquid suspensions, parenteral solutions, injections, tablets, pills, granules, powder, film, (micro)capsules, aerosols, tonics, syrups, beverages, or nourishments.
